(19) United States
(12) Patent Application Publication
(10) Pub. No.: US 2026/0354756 A1
(43) Pub. Date: Nov. 12, 2026

(54) SYSTEMS AND METHODS FOR MULTI-TIMESCALE METABOLIC CONTROL WITH INDEPENDENT SAFETY SUPERVISION

(71) Applicant: Steven B Sikes, Black Mountain, NC (US)

(72) Inventor: Steven B Sikes, Black Mountain, NC (US)

(21) Appl. No.: 19/645,162

(22) Filed: Apr. 11, 2026

(51) Int. Cl. A61B 5/00; A61B 5/145; A61M 5/142; A61M 5/172

(52) U.S. Cl. CPC A61B 5/4839; A61B 5/0004; A61B 5/14532; A61B 5/14546; A61B 5/7267; A61B 5/7275; A61B 5/7278; A61M 5/14248; A61M 2005/1726

(57) ABSTRACT

Systems and methods for multi-timescale metabolic control with independent safety supervision are disclosed. Short-timescale physiological measurement data are used to generate an internal metabolic state representation. Slower-evolving biological context information modifies adaptive update parameters that govern the behavior of an adaptive control framework without directly specifying therapeutic delivery amounts. The adaptive control framework generates a proposed therapeutic control action, which is validated using an independent safety supervision architecture prior to execution. In certain embodiments, safety bounds may be dynamically scaled based on uncertainty metrics, and asynchronous biological context signals may be temporally aligned using decay-weighted transformations. The architecture separates the temporal domains and preserves the independence between adaptive intelligence and safety enforcement, thereby enabling stable, modular, and extensible automated metabolic regulation systems.

### STATEMENT REGARDING FEDERALLY SPONSORED RESEARCH OR DEVELOPMENT

This invention was made without government support. No federal funds were used in the development of the disclosed systems or methods. The government has no rights to this invention.

### BACKGROUND OF THE INVENTION

### Field of the Invention

The present disclosure generally relates to automated metabolic regulation systems and, more particularly, to multi-timescale control architectures for adaptive therapeutic regulation with independent safety supervision.

Automated metabolic regulation systems, including closed-loop insulin delivery platforms, have advanced significantly in recent years. Many of these systems employ model predictive control, adaptive personalization algorithms, or reinforcement learning techniques to regulate glucose levels in individuals with diabetes.

Conventional systems typically rely on high-frequency physiological measurements, such as continuous glucose monitoring (CGM), to compute therapeutic delivery amounts in near real-time. Although these systems may incorporate adaptive tuning of control parameters, most operate within a single temporal control loop that treats all the input data as contemporaneous signals.

Emerging research suggests that metabolic regulation is influenced not only by short-term physiological dynamics but also by slower-evolving biological states, including biochemical, hormonal, inflammatory, and microbiome-associated factors. Existing systems often incorporate such data, if at all, as additional predictive features rather than structurally distinct regulatory signals.

Furthermore, many adaptive systems integrate safety constraints directly within the adaptive algorithm, potentially coupling safety enforcement to the internal model confidence or training state.

Thus, there remains a need for
Control architecture that explicitly separates fast metabolic dynamics from slower biological context signals,
Mechanisms for conditioning adaptive behavior based on slower-evolving biological states without directly dictating therapeutic output,
Safety supervision architectures that operate independently of adaptive learning logic,
Systems capable of integrating asynchronous multi-domain signals without destabilizing real-time control

The present disclosure addresses these issues.

### BRIEF SUMMARY OF THE INVENTION

The present disclosure provides systems and methods for multi-timescale metabolic control with independent safety supervision.

In one aspect, a computer-implemented method receives short-timescale physiological measurement data and generates an internal representation of the metabolic state. Slower-evolving biological context information is separately received and used to modify the adaptive update parameters that govern the behavior of an adaptive control framework. The adaptive control framework generates a proposed therapeutic control action based on an internal metabolic state representation.

Prior to execution, the proposed therapeutic control action is evaluated by an independent safety supervision architecture that enforces predefined clinical safety bounds.

In certain embodiments
Biological context information conditions adaptive learning behavior, rather than directly specifying therapeutic delivery.
Safety envelopes may be dynamically scaled based on uncertainty metrics.
Asynchronous biological signals may be temporally aligned using decay-weighted synchronization.
Control components may be implemented locally, remotely, or in distributed configurations.
Multiple metabolic modifying agents may be coordinated under the same architectural framework.

By structurally separating the temporal domains and preserving the independence between adaptive intelligence and safety enforcement, the proposed architecture improves the stability, modularity, and long-term adaptability of automated metabolic regulation systems.

### BRIEF DESCRIPTION OF THE DRAWINGS

FIG. 1—System Architecture of a Multi-Modal Automated Metabolic Regulation System

FIG. 1 illustrates an exemplary system architecture of an automated metabolic regulation system comprising a wearable therapeutic apparatus and associated components.

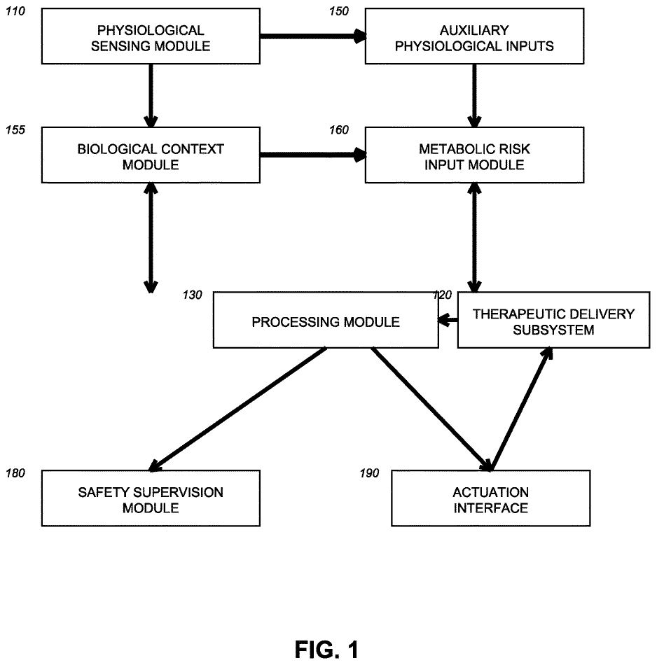

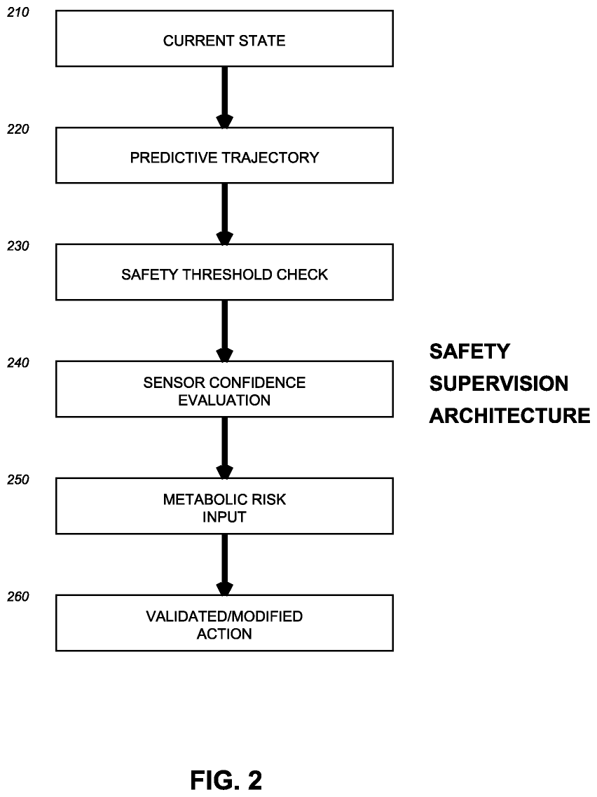

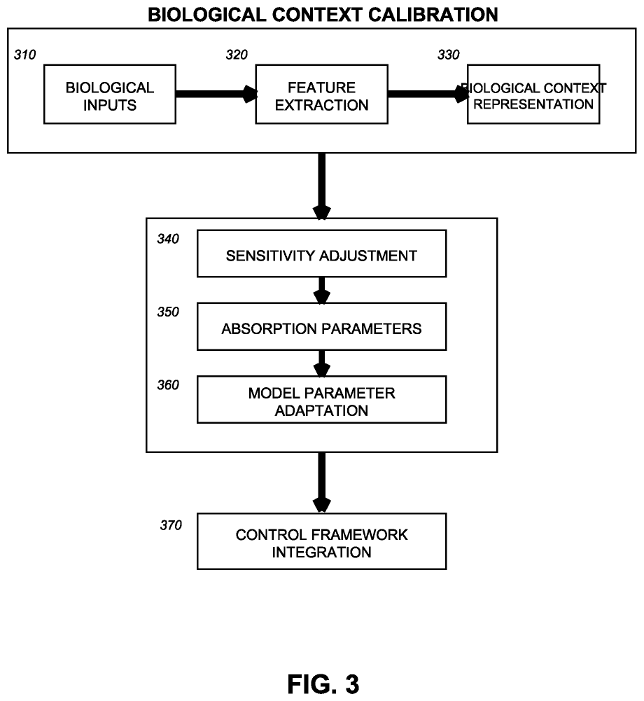

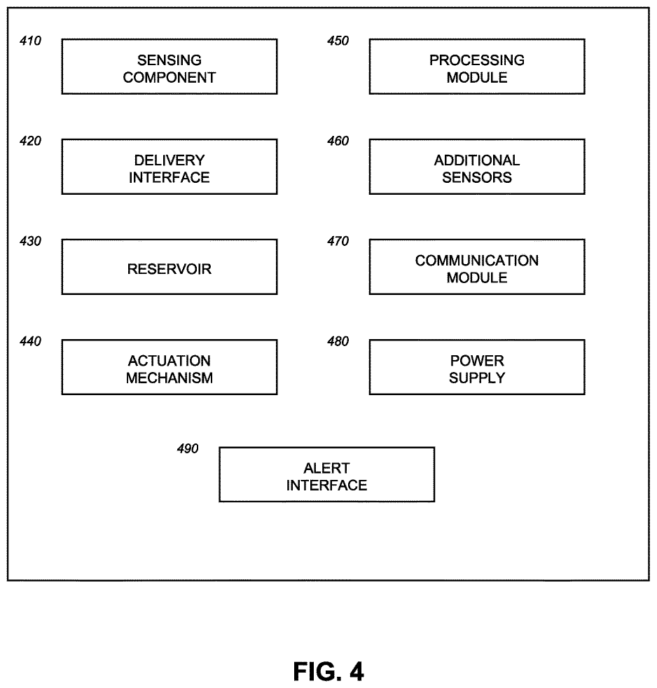

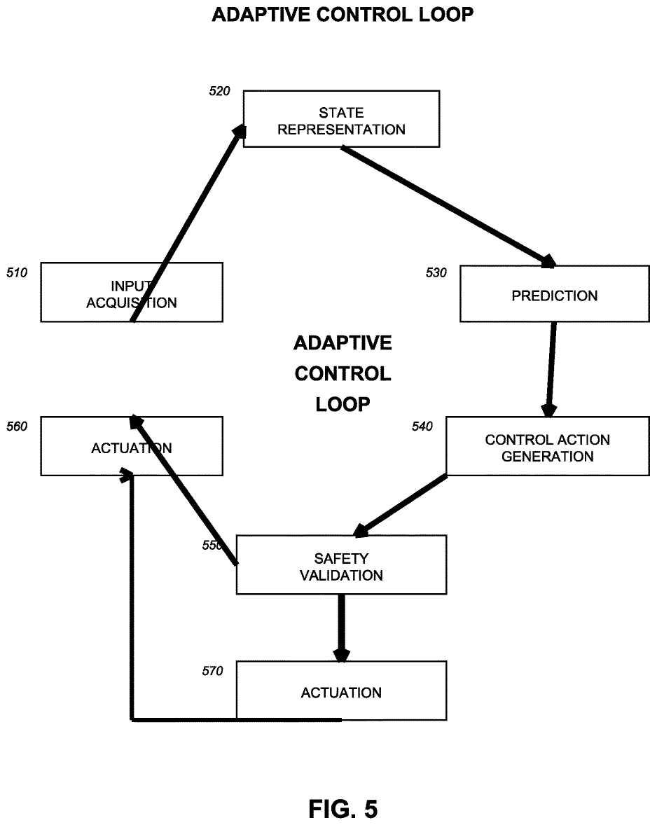

The system includes a continuous glucose monitoring (CGM) sensor (110) configured to generate glucose measurement data and a therapeutic delivery device (120) configured to deliver one or more metabolic-modifying agents.

A processing module (130), which may be integrated within the wearable therapeutic apparatus or communicatively coupled thereto, is configured to execute an adaptive control framework for generating proposed therapeutic control actions.

The system may further include one or more physiological sensing components (140) configured to acquire additional physiological signals, including, without limitation, heart rate, heart rate variability, activity level, sleep state, or temperature.

A biological context processing module (150) is configured to generate a biological context representation based on one or more biological inputs, which may include microbiome-derived information and other biochemical indicators.

A sensor confidence estimation module (160) is configured to determine a reliability metric associated with physiological measurement data.

A metabolic risk input module (170) is configured to receive additional metabolic risk indicators, including ketone measurements.

A safety supervision module (180) is configured to evaluate the proposed therapeutic control actions and enforce predefined safety constraints prior to execution.

An actuation interface (190) is configured to deliver validated therapeutic control actions to the therapeutic delivery device.

Communication between system components may be established via wired or wireless communication links (195).

FIG. 2—Safety Supervision Architecture for Therapeutic Action Validation

FIG. 2 illustrates an exemplary safety supervision architecture configured to validate the proposed therapeutic control actions prior to execution.

At a given time step (210), a predictive metabolic trajectory (220) is generated based on the current system state.

The safety supervision module evaluates whether the predicted metabolic values fall within the predefined safety thresholds (230). If a predicted value indicates the risk of a hypoglycemic event, a modified or inhibited therapeutic action is generated (235).

The safety supervision module further evaluates the sensor confidence metric (240). If the confidence metric indicates reduced reliability of the input data, the proposed therapeutic action may be scaled, constrained, or suspended (245).

The safety supervision module may also evaluate metabolic risk indicators (250), including ketone levels. If a risk condition is detected, a predefined safety protocol may be activated (255).

If no safety conditions are triggered, the proposed therapeutic control action is validated and forwarded for execution (260).

The safety supervision architecture operates independently of the adaptive control framework and is configured to intercept and validate therapeutic control actions prior to their actuation.

FIG. 3—Biological Context Calibration and Parameter Adaptation

FIG. 3 illustrates an exemplary biological context calibration process for conditioning an adaptive control framework.

Biological samples or inputs (310) are processed using a feature extraction module (320) to generate a biological context representation (330).

The biological context representation is used to modify one or more control-related parameters, including parameters associated with metabolic sensitivity (340), nutrient absorption characteristics (350), or predictive model parameters (360).

The calibrated parameters are provided to an adaptive control framework and associated safety supervision components (370), thereby influencing the system behavior without directly specifying the therapeutic control actions.

The biological context calibration process operates on a timescale that is slower than real-time physiological measurement processing.

FIG. 4—Wearable Therapeutic Apparatus Embodiment

FIG. 4 illustrates an exemplary embodiment of the automated metabolic regulation system implemented as a wearable therapeutic apparatus.

The wearable therapeutic apparatus may include a physiological sensing component (410) configured to obtain glucose measurements and a delivery interface (420) configured to administer one or more metabolic-modifying agents.

The apparatus may further include one or more reservoirs (430) for storing therapeutic agents and an actuation mechanism (440) configured to deliver controlled amounts of the therapeutic agents.

A processing module (450) is configured to execute control algorithms, including adaptive control and safety supervision functions.

Additional sensing components (460) may be included to acquire physiological signals.

A communication module (470) enables data exchange with external devices or remote systems.

A power supply (480) provides energy to the system components.

An alerting interface (490) may provide user feedback through visual, haptic, or auditory signals.

The wearable therapeutic apparatus may be configured as an adhesive on-body device or other wearable form factor.

FIG. 5—Adaptive Control Loop for Automated Metabolic Regulation

FIG. 5 illustrates an exemplary adaptive control loop for automated metabolic regulation.

At a first stage (510), physiological measurement data and contextual inputs are acquired.

At a second stage (520), a system state representation is generated based on the acquired inputs.

At a third stage (530), a predictive model estimates future metabolic behavior.

At a fourth stage (540), an adaptive control framework generates a proposed therapeutic control action.

At a fifth stage (550), a safety supervision module evaluates the proposed therapeutic control action.

At a sixth stage (560), a validated therapeutic control action is executed by a therapeutic delivery device.

At a seventh stage (570), system performance feedback is used to update internal control parameters.

The adaptive control loop operates continuously to regulate the metabolic state within predefined safety constraints.

### DETAILED DESCRIPTION OF THE EMBODIMENTS

### Wearable Therapeutic Apparatus and System Implementation

The following description sets forth exemplary embodiments of an automated metabolic regulation system implemented as a whole or in part as a wearable therapeutic apparatus. The embodiments are provided for illustration purposes and are not intended to limit the scope of the claimed subject matter.

### 1. Overview of Apparatus-Based Implementation

In certain embodiments, the automated metabolic regulation system is implemented as, or in conjunction with, a wearable therapeutic apparatus configured to acquire physiological data, process the data using one or more computational modules, and deliver one or more metabolic-modifying agents in accordance with validated therapeutic control actions.

The wearable therapeutic apparatus may be configured as an on-body device, including, but not limited to, an adhesive patch, wearable module, or integrated device configured for continuous or intermittent operation.

The apparatus may operate independently or in communication with one or more external computing devices, including mobile devices, wearable devices, or remote computing systems.

### 2. Physiological Sensing Components

In certain embodiments, the wearable therapeutic apparatus includes one or more physiological sensing components configured to obtain physiological measurement data from the subject.

Such sensing components may include a continuous glucose monitoring (CGM) sensor configured to generate glucose measurement data over time. In some embodiments, the CGM sensor may comprise a subcutaneous sensor, a transdermal sensor, or another minimally invasive or non-invasive sensing mechanism.

The apparatus may further include additional sensing components configured to acquire physiological signals, including, without limitation, heart rate, heart rate variability, motion or activity, temperature, sleep-related signals, or other physiological indicators.

Physiological measurement data can be acquired at regular intervals or continuously and provided to one or more processing modules for further analysis.

### 3. Therapeutic Delivery Components

In certain embodiments, the wearable therapeutic apparatus includes a therapeutic delivery subsystem configured to deliver one or more metabolic-modifying agents to a subject.

The therapeutic delivery subsystem may include one or more reservoirs configured to store therapeutic agents and an actuation mechanism configured to deliver controlled quantities of therapeutic agents.

In some embodiments, the therapeutic agent may include insulin. In other embodiments, the therapeutic agents may further include one or more additional agents, including counterregulatory hormones and adjunct therapeutic compounds.

The actuation mechanism may include a micropump, microneedle array, infusion system, or other delivery mechanisms capable of administering precise therapeutic doses.

The delivery of therapeutic agents is performed in accordance with the validated therapeutic control actions generated by the system.

### 4. Processing Module and Control Execution

The wearable therapeutic apparatus may include or be communicatively coupled to one or more processing modules configured to execute control logic associated with automated metabolic regulation.

The processing module may include one or more processors and associated memory configured to store instructions that, when executed, perform functions including:
generation of a system state representation based on physiological measurement data,
processing of contextual or auxiliary inputs,
generation of proposed therapeutic control actions, and
coordination with safety supervision components.

In certain embodiments, the processing module may execute an adaptive control framework configured to generate proposed therapeutic control actions that are responsive to changes in the physiological state.

The processing module may be implemented within the wearable therapeutic apparatus or distributed across one or more local or remote computing systems.

### 5. Biological Context Processing and Calibration

In certain embodiments, the system includes a biological context processing module configured to generate a biological context representation based on one or more biological inputs.

Such biological inputs may include, without limitation, microbiome-derived information, metabolomic indicators, hormonal signals, or other biochemical or physiological data associated with a longer-term metabolic state.

The biological context representation may be used to modify one or more control-related parameters or internal representations associated with the control framework.

In certain embodiments, the biological context processing module operates on a timescale that is slower than that of physiological measurement acquisition and control action generation.

The biological context processing module may be implemented within the wearable therapeutic apparatus or in a remote or distributed computing environment.

### 6. Sensor Confidence and Reliability Assessment

In certain embodiments, the system includes a sensor confidence estimation module configured to determine a reliability metric associated with the physiological measurement data.

The reliability metric may be derived based on one or more factors, including signal consistency, noise characteristics, calibration status, or comparison with expected physiological behavior.

The reliability metric may be used to influence processing decisions, including scaling, constraining, or suspending proposed therapeutic control actions when measurement confidence is reduced.

Sensor confidence estimation may be performed continuously or periodically during system operation.

### 7. Metabolic Risk Input Integration

In certain embodiments, the system includes a metabolic risk input module that is configured to receive additional metabolic risk indicators.

Such indicators may include ketone measurements obtained from blood, breath, or other measurement modalities or other biochemical markers indicative of metabolic risk conditions.

The metabolic risk input module provides input to the safety supervision architecture for evaluating risk conditions and determining appropriate safety responses.

### 8. Safety Supervision Architecture

A safety supervision module is configured to evaluate the proposed therapeutic control actions prior to execution and to enforce predefined safety constraints.

The safety supervision module may intercept the proposed therapeutic control actions generated by the control framework and evaluate such actions based on the predicted metabolic outcomes, sensor confidence metrics, and metabolic risk indicators.

If a proposed therapeutic control action is determined to violate safety constraints, the safety supervision module may modify, constrain, or prevent the execution of the action.

In certain embodiments, the safety supervision module operates independently of the adaptive control framework and does not require access to the internal control parameters or model structures.

The safety supervision module may be implemented locally within the wearable therapeutic apparatus or in a separate processing system.

### 9. Actuation Interface and Delivery Execution

An actuation interface is configured to receive validated therapeutic control actions and control the therapeutic delivery subsystem accordingly.

The actuation interface may translate control actions into device-specific commands for delivery of therapeutic agents.

In certain embodiments, the actuation interface operates only on control actions validated by the safety supervision module.

### 10. Communication and Distributed Operation

In certain embodiments, the components of the system may communicate via wired or wireless communication links.

The wearable therapeutic apparatus may communicate with external devices, including mobile computing devices, wearable devices, or remote computing systems.

Certain computational functions, including adaptive control computation or biological context processing, may be performed in distributed or remote environments.

Regardless of the computational distribution, therapeutic control actions may be subject to safety validation prior to execution.

### 11. User Feedback and Alerting

In certain embodiments, the wearable therapeutic apparatus includes an alerting interface configured to provide feedback to the user.

The alert interface may include visual, haptic, or auditory output mechanisms.

User feedback may be generated in response to system state, safety events, or operational conditions.

### 12. Alternative Embodiments and Configurations

The components described herein may be implemented in various configurations, including fully integrated wearable devices, partially distributed systems, or combinations thereof.

The wearable therapeutic apparatus may be configured in different physical form factors and may incorporate additional or alternative sensing, processing, or delivery components.

The described embodiments are illustrative and not limiting, and modifications may be made without departing from the scope of the claimed subject matter.

### System Architecture for Adaptive Interaction and Control

### 2. Short Description

This figure illustrates the overall system architecture, including the physiological inputs, interaction inputs, state representation, causal attribution, coordinated intervention, and safety supervision modules.

1. A wearable therapeutic apparatus for automated metabolic regulation of a subject, comprising:
one or more physiological sensing components configured to generate physiological measurement data including at least glucose measurement data;
a therapeutic delivery subsystem configured to deliver one or more metabolic-modifying agents to the subject;
one or more processing modules configured to:
generate a system state representation based on the physiological measurement data;
generate a proposed therapeutic control action based on the system state representation using an adaptive control framework;
receive a biological context representation indicative of a metabolic state of the subject; and
condition at least one of a parameter, constraint, or internal representation of the adaptive control framework based on the biological context representation;
a safety supervision module configured to evaluate the proposed therapeutic control action based on one or more safety criteria and to generate a validated therapeutic control action;
and an actuation interface configured to control the therapeutic delivery subsystem based on the validated therapeutic control action,
wherein the safety supervision module is configured to intercept and evaluate the proposed therapeutic control action prior to execution by the therapeutic delivery subsystem.

2. A system for automated metabolic regulation comprising:
a wearable therapeutic apparatus including a physiological sensing component and a therapeutic delivery subsystem; and
one or more processing systems communicatively coupled to the wearable therapeutic apparatus and configured to:
generate a system state representation based on physiological measurement data received from the wearable therapeutic apparatus;
generate a proposed therapeutic control action using an adaptive control framework;
receive a biological context representation and condition at least one of a parameter, constraint, or internal representation of the adaptive control framework based on the biological context representation; and
validate the proposed therapeutic control action using a safety supervision module prior to execution, wherein the wearable therapeutic apparatus is configured to deliver a therapeutic agent based on the validated therapeutic control action.

3. The apparatus of claim 1, wherein the biological context representation is derived from microbiome-related data.

4. The apparatus of claim 1, wherein the conditioning comprises adjusting a parameter associated with insulin sensitivity.

5. The apparatus of claim 1, wherein the conditioning comprises modifying a control constraint associated with therapeutic delivery limits.

6. The apparatus of claim 1, wherein the biological context representation is updated at a timescale slower than that of the generation of the proposed therapeutic control action.

7. The apparatus of claim 1, wherein the adaptive control framework comprises a learning-based control system configured to adapt over time.

8. The apparatus of claim 7, wherein the learning-based control system comprises a reinforcement-learning-based controller.

9. The apparatus of claim 1, wherein the adaptive control framework operates in conjunction with the baseline control mechanism.

10. The apparatus of claim 1, wherein the safety supervision module is configured to modify or inhibit the proposed therapeutic control action when a predicted metabolic condition exceeds the safety threshold.

11. The apparatus of claim 1, wherein the safety supervision module operates independently of the internal parameters of the adaptive control framework.

12. The apparatus of claim 1, wherein the safety supervision module is configured to evaluate a reliability metric associated with the physiological measurement data.

13. The apparatus of claim 12, wherein the reliability metric is based on a signal noise, consistency, or calibration status.

14. The apparatus of claim 1, wherein the proposed therapeutic control action is scaled or constrained based on a reliability metric associated with physiological measurement data.

15. The apparatus of claim 1, further comprising a metabolic risk input module configured to receive a metabolic risk indicator.

16. The apparatus of claim 15, wherein the metabolic risk indicator comprises a ketone measurement.

17. The apparatus of claim 15, wherein the safety supervision module modifies or inhibits the proposed therapeutic control action based on the metabolic risk indicators.

18. The apparatus of claim 1, wherein the therapeutic delivery subsystem comprises a micropump or microneedle delivery mechanism.

19. The apparatus of claim 1, wherein the metabolic-modifying agent comprises insulin.

20. The apparatus of claim 1, wherein the therapeutic delivery subsystem is configured to deliver multiple therapeutic agents.

21. The apparatus of claim 1, wherein at least a portion of the processing modules is implemented on a remote computing system.

22. The apparatus of claim 1, wherein the apparatus communicates with an external device via wireless communication.

23. The apparatus of claim 1, wherein the wearable therapeutic apparatus comprises an adhesive on-body device.

24. The apparatus of claim 1, wherein the wearable therapeutic apparatus comprises an implantable device.

25. The method of claim 1, wherein the adaptive control framework generates therapeutic control actions for a plurality of metabolic-modifying agents under a common multi-timescale conditioning and safety supervision architecture
